(19) United States
(12) Patent Application Publication
(10) Pub. No.: US 2021/0380919 A1
(43) Pub. Date: Dec. 9, 2021

(54) LASER ASSISTED METAL ADHESION TO INDIUM TIN OXIDE ON GLASS, QUARTZ, SAPPHIRE AND SINGLE CRYSTAL SILICON WAFER SUBSTRATES FOR HEATED PLATFORMS FOR CELL CULTURING

(71) Applicant: KING ABDULLAH UNIVERSITY OF SCIENCE AND TECHNOLOGY, Thuwal (SA)

(72) Inventor: Ulrich BUTTNER, Thuwal (SA)

(21) Appl. No.: 17/283,075

(22) PCT Filed: Jul. 24, 2019
(86) PCT No.: PCT/IB2019/056331

§ 371 Date: Apr. 6, 2021

Related U.S. Application Data
(60) Provisional application No. 62/744,187, filed on Oct. 11, 2018.
(60) Provisional application No. 62/807,528, filed on Feb. 19, 2019.
(60) Provisional application No. 62/835,751, filed on Apr. 18, 2019.

(51) Int. Cl. C12M 3/06; B81C 1/00; C12M 1/00; C12M 1/02; B23K 26/324; B23K 26/12; B23K 26/08

(52) U.S. Cl. CPC C12M 23/16; B81C 1/00095; C12M 23/20; C12M 47/02; B81C 2201/0188; C12M 41/22; B23K 26/324; B23K 26/123; B23K 26/08; C12M 23/22

(57) ABSTRACT

A method for directly bonding a metal to a transparent substrate includes providing a substrate; placing a metal foil directly on a face of the substrate; irradiating a portion of the metal foil with a laser beam so that metal corresponding to the portion melts and bonds directly to the substrate and forms a metal pad; and pumping a gas above the portion to prevent oxidation of the melted metal.

### CROSS-REFERENCE TO RELATED APPLICATIONS

This application claims priority to U.S. Provisional Patent Application No. 62/744,187, filed on Oct. 11, 2018, U.S. Provisional Patent Application No. 62/807,528, filed on Feb. 19, 2019, and U.S. Provisional Patent Application No. 62/835,751, filed on Apr. 18, 2019, all entitled “LASER ASSISTED METAL ADHESION ON ITO, GLASS AND SILICON WAFERS,” the disclosures of which are incorporated herein by reference in their entirety.

### BACKGROUND

### Technical Field

Embodiments of the subject matter disclosed herein generally relate to making a metal pad on a transparent or Si based substrate, and more specifically, to quickly bonding a metal pad to a transparent substrate without strenuous conditions. In one embodiment, a metal contact is attached to a transparent conductor, such as a thin film of Indium Tin Oxide (ITO) on Glass, Quartz, Sapphire or Si and then a voltage is applied to generate heat to keep a cell culture alive over an extended period of time.

### Discussion of the Background

Recently, with the advance of miniature electronics, there is a movement to develop microfluidic platforms for in vitro cell studying. These platforms need an array of sensors to be placed next to the cells so that the sensors can directly monitor the cells and their environment. Further, there is a desire to observe the cells with various imaging devices and these devices need optical access to the cells.

For these reasons, the new microfluidic platforms are made of a transparent substrate and in general of transparent materials so that light can enter the cavity where the cells are held, interact with the cells, and then reflect back so that information about the cells is collected by imagining devices. However, the new microfluidic platforms also need electrical contacts to the various components and sensors that are embedded into the platforms.

It is known in the art to adhere an electrical pad onto a transparent substrate, for example, glass. Such a process uses a sputter deposition method, which is a physical vapor deposition (PVD) method of thin film deposition. This method places a substrate (the transparent substrate in this case) into a chamber in which the air is evacuated. On an electrode opposite to the substrate, the target material (copper in this case) is placed. A high voltage is then applied between the target material and the chamber, an inert gas such as Argon is supplied and in the presence of a magnetic field, Argon ions are created. These ions are accelerated onto the target material and thereby dislodging atoms, which deposit onto to the transparent substrate. A similar process that is used for depositing an electric pad on a substrate is electron beam metal evaporation. After the thin layer of copper (electric pad) is deposited onto the substrate, the entire assembly enters a high-temperature annealing stage, where stress is removed from the newly formed device and the adhesion properties are improved.

However, these methods require the creation of high vacuum, need high-energy for evaporating the metal, and also they are time intensive. Thus, there is a need for a new method, for improving the metal pad adhesion, to a transparent substrate that overcome the above noted deficiencies.

### SUMMARY

According to an embodiment, there is a method for directly bonding a metal to a transparent substrate. The method includes providing a substrate, placing a metal foil directly on a face of the substrate, irradiating a portion of the metal foil with a laser beam so that metal corresponding to the portion melts and bonds directly to the substrate and forms a metal pad, and pumping a gas above the portion to prevent oxidation of the melted metal.

According to another embodiment, there is a microfluidic platform for growing cells. The microfluidic platform includes a silicon wafer having microfluidic passages in which the cells grow, a glass layer formed directly on a first face of the silicon wafer, a first indium tin oxide, ITO, layer formed directly on the glass layer, opposite to the silicon wafer, and first and second metal pads form directly on the first ITO layer. The first and second metal pads are connected to a power source so that the first ITO layer acts as a heater for heating the microfluidic passages.

According to still another embodiment, there is a method of making a microfluidic platform that is entirely transparent to electromagnetic waves. The method includes forming microfluidic passages in a silicon wafer, forming a first indium tin oxide, ITO, layer directly on a first glass layer, attaching the first glass layer directly to a first face of the silicon wafer, and forming first and second metal pads directly onto the first ITO layer.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 illustrates a system for bonding a metal pad directly to a transparent substrate with a laser beam;

FIG. 2 illustrates the formed metal pad on the transparent substrate;

FIG. 3 is a flowchart of a method for laser assisted bonding a metal pad to a transparent substrate;

FIG. 4 illustrates another system for bonding a metal pad directly to a transparent substrate with a laser beam;

FIG. 5 illustrates a microfluidic platform that uses metal pads bonded directly to a transparent substrate;

FIG. 6 illustrates the various layers of the microfluidic platform, separated from each other;

FIG. 7 illustrates another microfluidic platform that uses metal pads bonded directly to a transparent substrate;

FIG. 8 illustrates the various layers of this microfluidic platform, separated from each other;

FIG. 9 illustrates a growing cell system that uses a microfluidic platform having metal pads directly formed on a transparent substrate;

FIG. 10 illustrates various components of the growing cell system;

FIG. 11A illustrates wires soldered to metal pads, which were formed directly on a glass substrate and FIG. 11B illustrates a transparent substrate that has various sensors;

FIG. 12 illustrates neuron cells growing in the microfluidic platform;

FIG. 13 is a flowchart of a method for laser assisted metal bonding to a transparent substrate; and

FIG. 14 is a flowchart for making a growing cell system that uses a microfluidic platform.

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to depositing a metal pad on a Si or SiO2 (glass) transparent substrate. However, the methods discussed herein can also be applied to depositing metal structures more complicated than a simple pad, and/or to depositing a metal pad on a substrate that is not transparent.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a metal pad is deposited on a transparent substrate at atmospheric pressure. A thin foil of the metal to be deposited is placed on the transparent substrate, which may include glass, quartz, sapphire, silicon wafer, or a thin film of Indium Tin Oxide (ITO) material deposited on these substrates. A laser is directed on a portion of the foil and simultaneously, an inert gas is blown over the same portion to prevent oxygen from the air to oxidize the metal, while the metal is being melted by the laser beam. The portion of the metal foil that is irradiated with the laser beam is melted and directly bonded to the transparent substrate or silicon wafer. The laser can be moved along the metal foil to pattern the metal pad as desired. No annealing is necessary after the metal has been bonded to the transparent glass. The metal foil left on top of the substrate is removed at the end of the process of bonding the metal to the substrate.

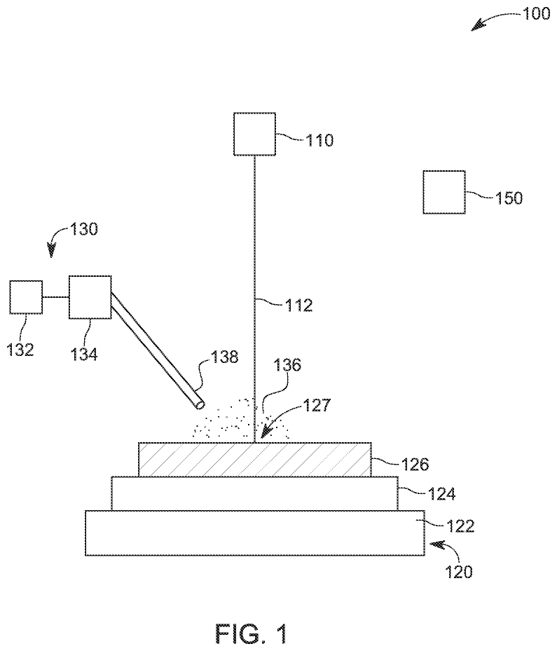

This method is now discussed in more detail with regard to the figures. FIG. 1 shows a system 100 that includes a laser device 110, a substrate 120, and a gas supply system 130. The laser device 110 may be an Ytterbium Fiber Laser having a power of about 40 W and a wavelength of about 1060 nm. Note that other types of lasers, having different powers may be used. The wavelength may be in the range of 400 nm to 20,000 nm. Depending on the frequency of the laser, if the laser is a pulsed laser, the energy delivered by the beam 112 onto a unit area of the substrate 120, i.e., the fluence, needs to be high enough so that the part of the metal foil that needs to be deposited on the substrate is melted. Thus, depending on the metal that makes up the foil, the fluence of the laser used can be calculated and the laser can be adjusted to generate such fluence.

The substrate 120 shown in FIG. 1 includes a transparent portion, which can be glass 122 or ITO 124 or both. FIG. 1 shows the ITO 124 formed over the glass 122. However, in one embodiment, only one of the ITO 124 or glass 122 is present. On top of the substrate 120, there is placed the metal foil 126, from which the metal pad is going to be formed. The gas supply system 130 includes a gas storage unit 132, which is an inert gas pressurized cylinder, which is fluidly connected to a pump 134. The pump 134 blows the gas 136 from the gas storage unit 132 onto a portion 127 of the metal foil 126, to prevent oxidation of that portion. A nozzle 138 may be used to direct the gas 136 to the portion 127, where the laser beam 112 interacts with the metal foil 126. The gas 136 may be an inert gas, as for example, Ar, N, He, Ne, Kr, Xe, CO2 or a combination of them.

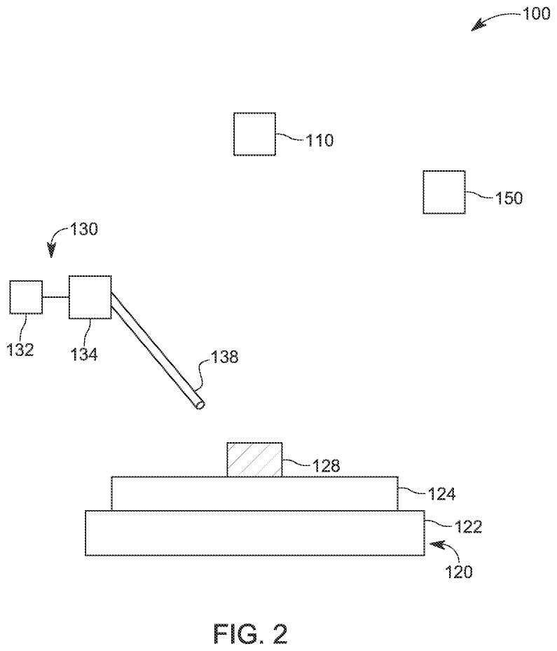

A controller 150 is connected to the laser 110 and to the gas supply system 130 for controlling (1) the fluence gas flow to the laser beam, and (2) when to turn on and off the laser beam. The controller can be programmed to adjust the flowing of the gas when the laser beam from the laser is melting the metal foil. Also, the controller 150 may be programmed to move the laser beam along a given pattern to form the metal pad. Alternatively, the controller may be configured to move the laser or the substrate 120 to form the desired shape of the metal sheet design (pad). After the laser beam has melted a part of the metal foil at the portion 127 to form a metal pad 128, as shown in FIG. 2, the remaining part of the metal foil can be removed, the gas supply system is stopped, and the laser is switched off. Note that no other material or layer has been formed between the substrate 120 and the metal pad 128, i.e., the metal pad 128 has been formed/bonded directly to the substrate 120.

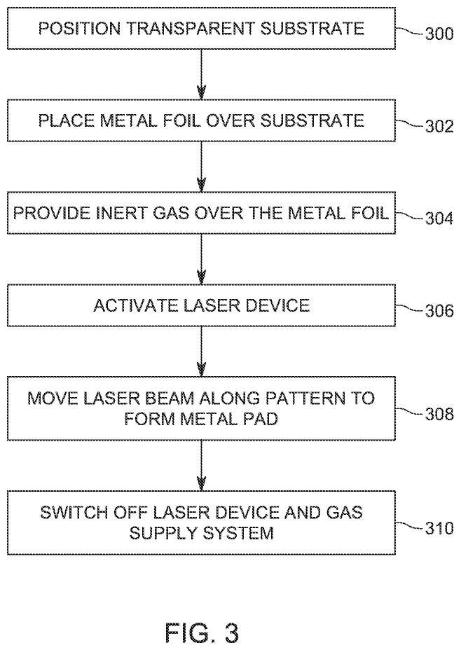

Having the metal pad 128 strongly bonded to the substrate, it is now easy to solder an electrical connection to it. The process of depositing the metal pad on the transparent substrate discussed above is quick, requires a low amount of energy, and does not require vacuum conditions. This process is described now with regard to FIG. 3. In step 300, a transparent substrate, or a silicon wafer is positioned to be impacted by a laser beam. In step 302, a metal foil is placed over the substrate, at a location where a metal pad is to be placed, close to the surface to be bonded, on the substrate. In step 304, an inert gas is provided above the metal foil, where the laser beam is expected to interact with the metal foil, to prevent oxidation. In step 306 a laser device is activated to generate a laser beam and a fluence of the laser beam is adjusted to melt the metal from the metal foil. In step 308, the laser beam is moved along a given path to form the metal pad with a desired shape or the substrate is moved relative to the laser beam to achieve the same result. In step 310, the laser device is switched off and the gas supply system is switched off.

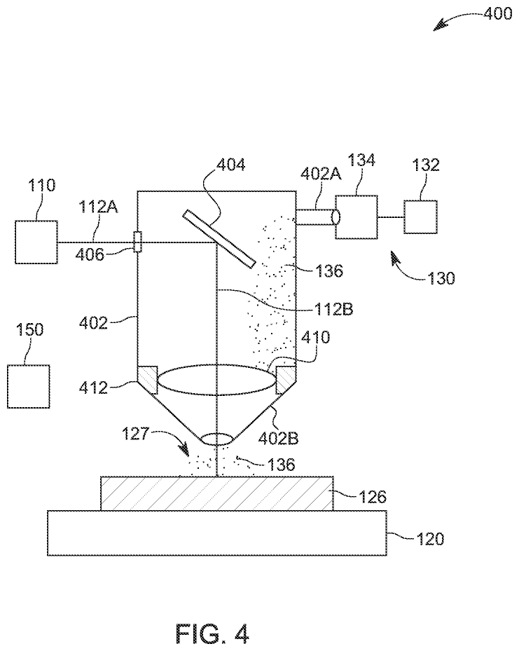

FIG. 4 shows another setup 400 for forming a metal pad on a transparent substrate. In this configuration, there is an enclosure 402 that holds a mirror 404. The laser beam 112A enters the enclosure 402 through a transparent port 406, is reflected by the mirror 404, and then the reflected laser beam 1128 is focused with a focusing lens 410 onto the top portion of the metal foil 126. The enclosure 402 has an input 402A that is fluidly connected to the pump 134 of the gas supply system 130. Thus, the gas 136 flows through the enclosure 402 and exists at output 402B, directly onto the metal foil 126, to prevent an oxidization of the portion 127.

A focusing lens 410 may be placed inside the enclosure 402 and may be moved up and down with a moving mechanism 412, which is coordinated by the controller 150. The focusing lens 410 is adjusted up or down so that the laser beam 1128, which is reflected on the mirror 404, is focused on the desired portion 127 on top of the metal foil 126. This means that depending on the thickness of the substrate 120 and the thickness of the metal foil 126, the focusing lens 410 needs to adjust the focusing point of the laser beam 4128. In one application, a thickness of the metal foil is between 10 and 30 μm. A thickness of the substrate 120 can have any value. A size of the metal foil 126 may be about 20 to 50 μm. The metal pad may be made to be a square, rectangle or any other shape. A thickness of the metal pad 128 may be made to be between 20 and 50 μm, with a preferred range of 20 to 30 μm. Those skilled in the art would understand that other configurations may be used as long as a laser beam impinges on the metal foil, and the oxygen is removed from where the laser beam melts the metal to prevent oxidation.

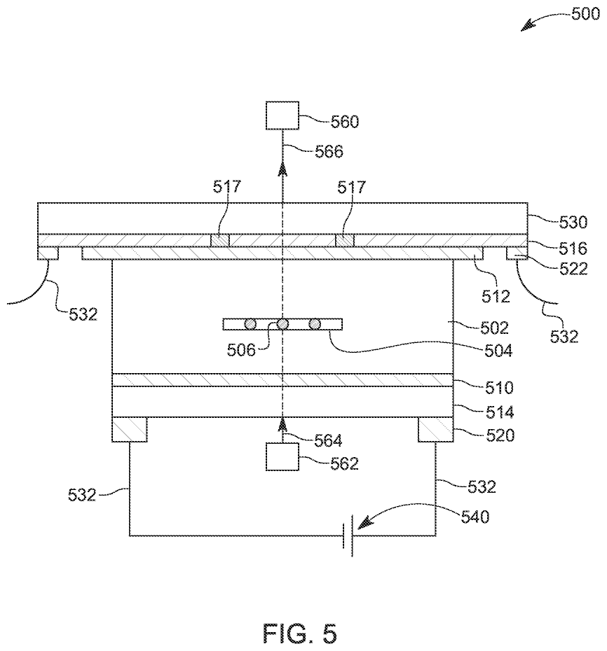

The technique discussed with regard to FIG. 3 can be applied to build a microfluidic platform for integrating sensors for in vitro cell probing. For example, as illustrated in FIG. 5, a microfluidic platform 500 includes a Si layer 502 having plural microfluidic channels 504 (only one shown in the figure for simplicity) in which one or more cells 506 are grown. Fluids with nutrients (not shown) may be supplied to the microfluidic channels. The Si layer 502 may be formed on a thin glass bottom layer 510, which is transparent to light, as is the Si layer 502.

On top of the Si layer 502, a gasket made of polydimethylsiloxane (PDMS) material 512 may be placed to prevent the fluid from the channels 504 to escape outside the Si layer. A thin glass top layer 530 is placed at the top part of the platform 500. One or more sensors 517 are formed in the ITO material 516, which are also transparent to light. The ITO material 516 may be deposited directly on the glass top layer 530. Metal pads 522 are formed on the ITO material 516 as discussed with regard to FIG. 3.

In one application, a thin film heater 514, for example, made from transparent ITO material, is formed on the glass bottom layer 510, for heating the fluid and cells inside the channels 504. Metal pads 520 for the heater layer 514 are formed directly on the ITO material, as discussed with regard to FIG. 3. Wires 532 may be soldered to all these metal pads to connect them to the controller 150, and/or to a power source 540. The power source 540 may be a battery, fuel cell, AC outlet, etc. The wires 532 may also be connected to other devices.

Because all the layers discussed herein are transparent to light, an imaging device 560 may be provided on one side of the platform 500 and a source of light 562 may be provided on the other side of the platform for generating a light beam 564. The light beam 564 may traverse all the layers of the platform 500, may interact with one or more cells 506, and thus, the output light 566 may carry information about the cells. The output light 566 is received by the imaging device 560 and analyzed for producing, for example, an image of the cells. The imaging device 560 may be a microscope, spectroscope, MRI machine, CT machine, X-ray machine, etc.

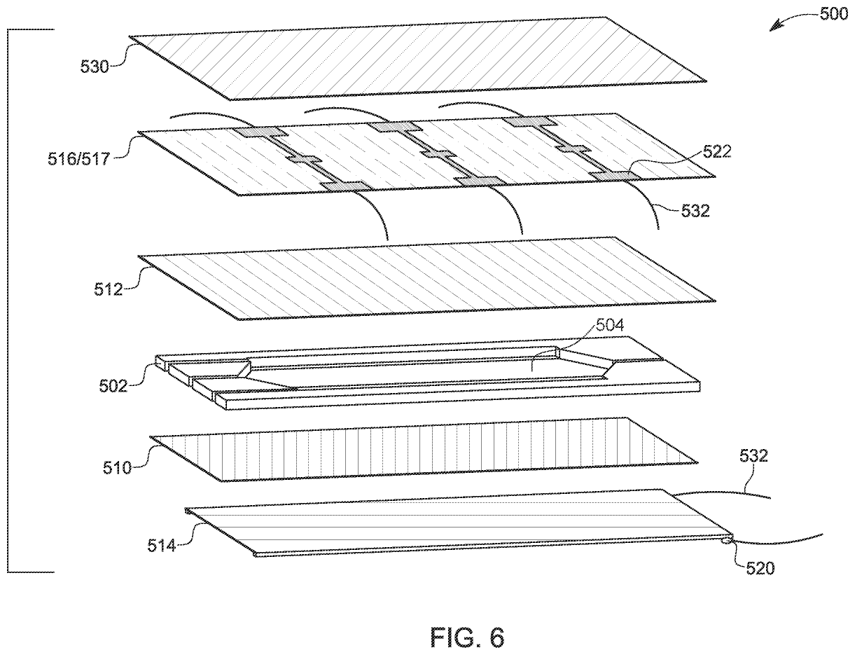

FIG. 6 shows an exploded view of the microfluidic platform 500. The various sensors 517 may include resistor or capacitance sensors. The ITO layer 516 may be any transparent and conductive thin layer, for example, a layer coated onto the glass layer 530 and etched with the laser to form the metal pads and the sensors. The PDMS layer 512 allows the chip to be opened after usage. The metal pads 522, formed for example, from copper, brass, bronze or aluminum, are fabricated with the laser assisted metal bonding discussed above with regard to FIG. 3.

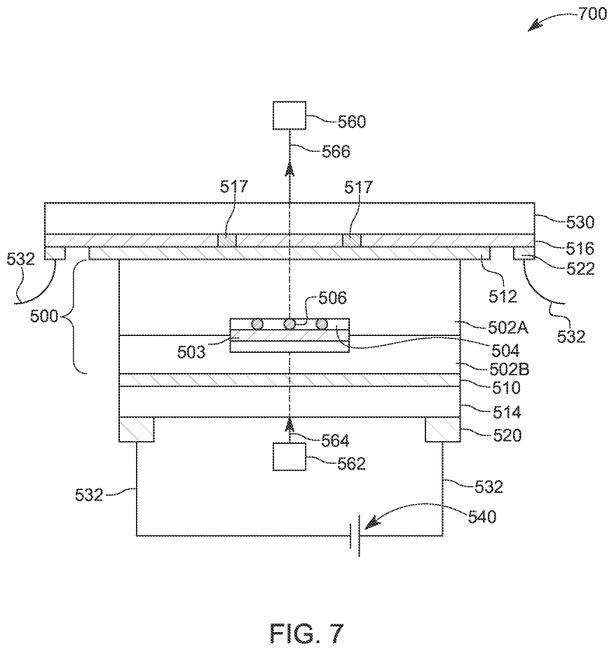

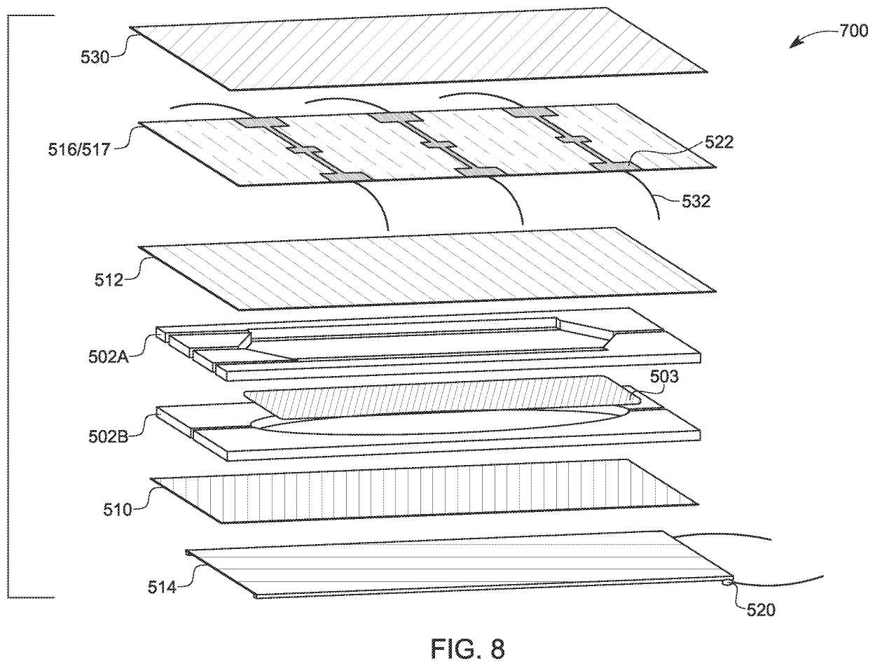

FIG. 7 shows a variation of the microfluidic platform 500 of FIG. 5, in which the Si layer 502 is split into an upper silicon microfluidic channel 502A and a lower silicon microfluidic channel 502B that are separated from each other by a microporous membrane 503. This configuration 700 is shown in FIG. 8 in an exploded view. The microporous membrane 503 is used to grow the cells and separate the cells from the fluids. For example, the upper Si layer 502A may be used to house the cells while the lower Si layer 502B is used to flow the nutrients. The nutrients are capable of diffusing through the porous membrane toward the cells while the cells cannot move through the membrane. In other words, the nutrients migrate through the microporous membrane to the upper Si layer 502A to feed the cells while the cells are hold in the upper Si layer 502A and cannot enter the lower Si layer 502B.

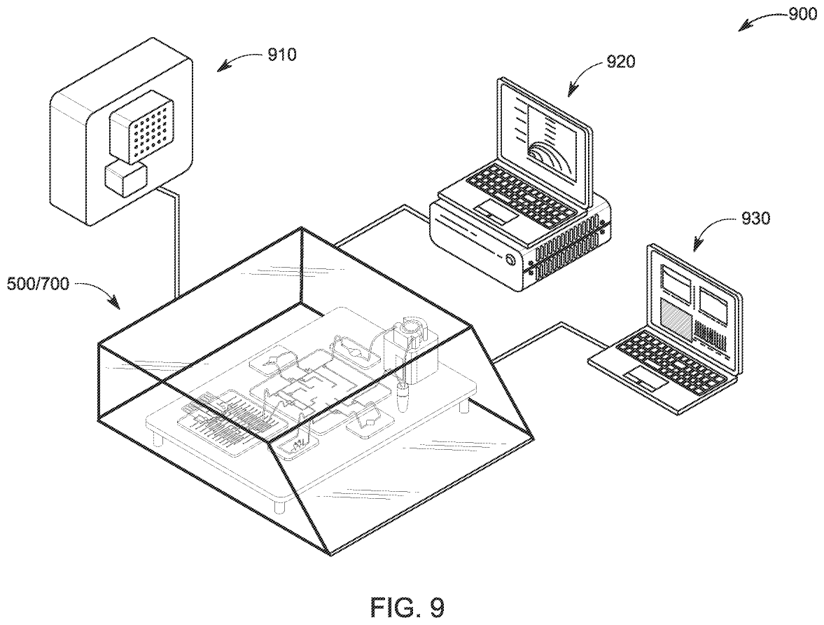

The platform 500 or 700 has been integrated into a known automatic system 900 for growing organs as illustrated in FIG. 9. The automatic system 900 includes the platform 500 or 700, an automated valve controller 910, a biomarker detection system 920, and an extracellular microenvironment monitoring system 930. The automated valve controller 910 is configured to regulate an amount of fluid and/or nutrient that flows into the platform 500 or 700. The fluid may include the nutrients necessary for the cells to grow a desired organ. The biomarker detection system 920 monitors various parameters associated with the cell grow so that an organ can be generated. For example, an amount of albumin in the microfluidic channels of the platform 500 or 700 need to have a certain profile during the growth of the organ. Thus, the biomarker detection system 920 monitors the presence of albumin during this process and display this and other biomarkers to the operator of the system.

The extracellular microenvironment monitoring system 930 is connected to the various sensors 517 formed in the platform 500 or 700 and monitors various physical and/or chemical parameters associated with the organ growing. For example, the system 930 is connected to a resistor sensor 517 for determining the temperature of the medium in which the cells are growing and is programmed, if the temperature is too low, to provide power to the heater 514 to increase the temperature of the cells. Other physical and/or chemical parameters may be monitored with the sensors 517, as for example, a pH of the medium.

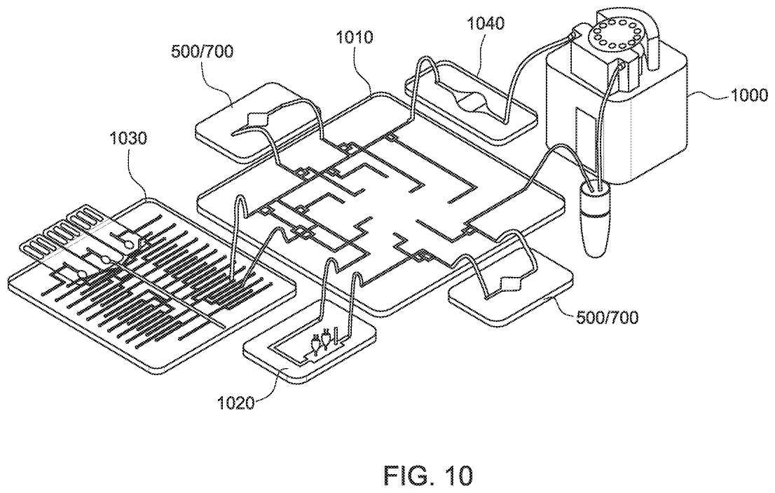

The platform 500 or 700 is shown in more detail in FIG. 10 and may include a pump 1000, which simulates the peristaltic conditions that are taking place in a real organism. The pump 1000 is fluidly connected to an automated flow control breadboard 1010 that is connected to one or more organ modules (i.e., platforms 500 or 700). A physical/chemical sensing module 1020 may be fluidly connected to the automated flow control breadboard 1010 for performing parameter measurements associated with the fluid flowing through these units. A bio-electrochemical sensing module 1030 may also be fluidly connected to the automated flow control breadboard 1010 for performing detection of various chemicals present in the fluid that is circulating to the growing organ. To prevent air to enter the pump 1000, a bubble trap device 1040 may be added to the return of the pump.

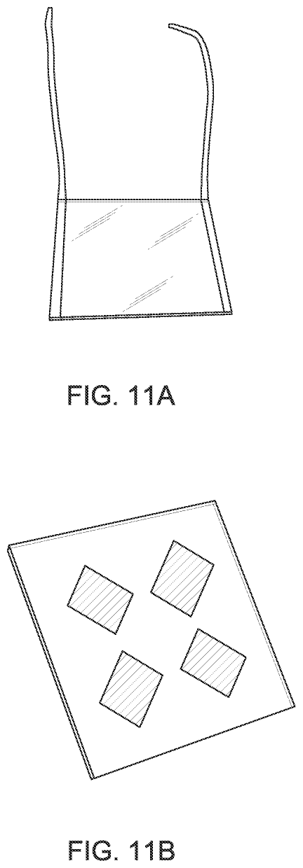

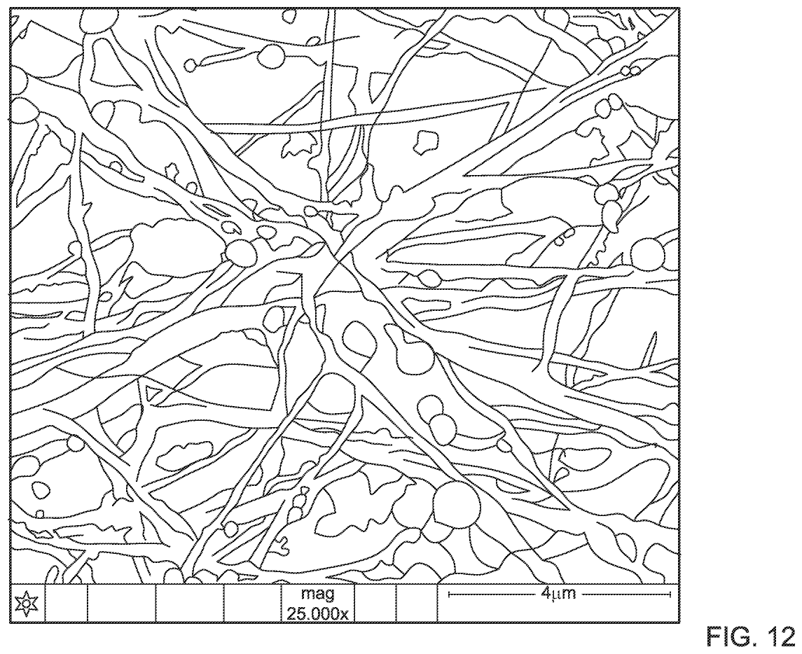

To exemplify some of the novel features that were added to an existing automatic growing system, to obtain the automatic growing system 900 illustrated in FIG. 9, FIG. 11A shows an ITO layer (having a thickness of 300 nm) to which two copper strips were added using the metal pads formed in FIG. 2. FIG. 11B illustrates a four chamber interdigitated ITO sensor that corresponds to elements 516/517 in FIG. 5. With the growing system 900, it is possible to grow neuron cells, as shown in FIG. 12, or other types of cells in real time, on a silicon platform that can mimic the conditions found inside the human body. In addition, due to the transparency of most of the elements that make the platform 500, it is now possible to observe live the cell growth.

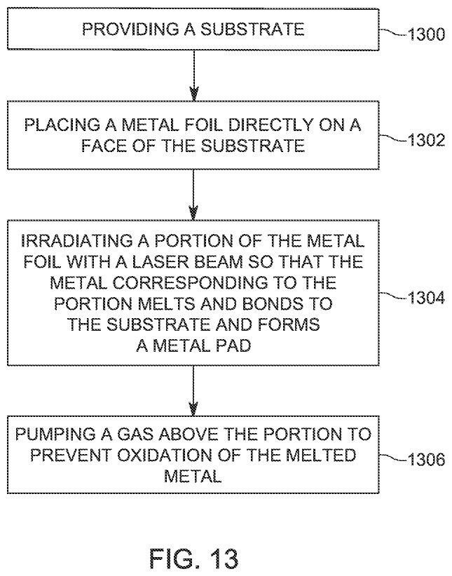

A method for directly bonding a metal to a transparent substrate is now discussed with regard to FIG. 13. The method includes a step 1300 of providing a substrate, a step 1302 of placing a metal foil directly on a face of the substrate, a step 1304 of irradiating a portion of the metal foil with a laser beam so that the metal corresponding to the portion melts and bonds to the substrate and forms a metal pad, and a step 1306 of pumping a gas above the portion to prevent oxidation of the melted metal. The gas may be purged before and during the laser beam irradiation. The metal foil includes at least one of copper, bronze, brass, aluminum, gold, silver, mild steel, zinc, tin and titanium. The transparent substrate includes at least one of glass, quartz, Sapphire, silicon, and indium tin oxide thin film, coated on these transparent substrates. The method may further include a step of controlling the fluence of the laser beam with a controller, so that the metal melts, and/or moving the laser beam along the metal foil to obtain a desired shape of the metal pad, and/or soldering an electrical wire to the metal pad, and/or guiding the laser beam with a mirror through a housing before arriving at the portion of the metal foil, and/or pumping the gas into the housing so that the laser beam and the gas exit from the housing at the same output. In one application, the output of the housing guides the laser beam and the gas directly onto the portion of the metal foil.

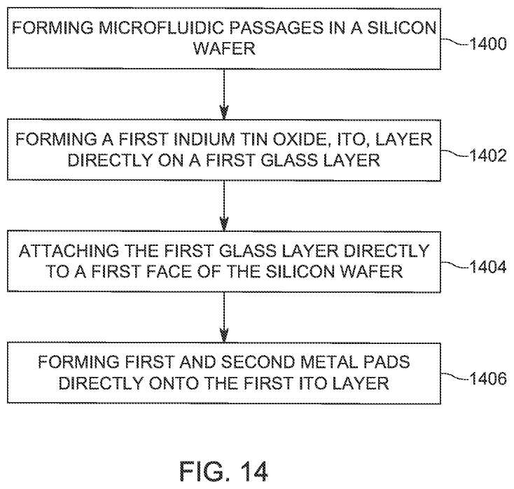

The platform 500 or 700 discussed above may be manufactured as now discussed with regard to FIG. 14. A method of making a microfluidic platform that is entirely transparent to electromagnetic waves includes a step 1400 of forming microfluidic passages in a silicon wafer 502, a step 1402 of forming a first indium tin oxide, ITO, layer 514 directly on a first glass layer 510, a step 1404 of attaching the first glass layer 510 directly to a first face of the silicon wafer 502, and a step 1406 of forming first and second metal pads 520 directly onto the first ITO layer 514. The method further includes a step of forming a second ITO layer 516 onto a second glass layer 530, a step of attaching a layer of polydimethylsiloxane, PDMS, 512 directly to a second face of the silicon wafer 502, which is opposite to the first face, and a step of attaching the second ITO layer directly to the PDMS layer. The method may further include a step of forming metal pads 522 directly onto the second ITO layer by melting a metal foil on the second ITO layer with a laser beam, while pumping an inert gas where the laser beam interacts with the metal foil to prevent oxidation, as discussed with regard to FIG. 3.

The disclosed embodiments provide a novel method for forming a metal pad directly on a transparent substrate and also a novel platform for growing cells. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A method for directly bonding a metal to a transparent substrate, the method comprising:
providing a substrate;
placing a metal foil directly on a face of the substrate;
irradiating a portion of the metal foil with a laser beam so that metal corresponding to the portion melts and bonds directly to the substrate and forms a metal pad; and
pumping a gas above the portion to prevent oxidation of the melted metal.

2. The method of claim 1, wherein the metal foil includes at least one of copper, bronze, brass, gold, silver, titanium, mild steel, Zinc, Tin and aluminum.

3. The method of claim 1, wherein the transparent substrate includes at least one of glass, quartz, Sapphire, silicon, and indium tin oxide.

4. The method of claim 1, further comprising:
controlling a fluence of the laser beam with a controller so that the metal melts.

5. The method of claim 4, further comprising:
moving the laser beam along the metal foil to obtain a desired shape of the metal pad.

6. The method of claim 1, wherein the gas is an inert gas.

7. The method of claim 1, further comprising:
soldering an electrical wire to the metal pad.

8. The method of claim 1, further comprising:
guiding the laser beam with a mirror through a housing before arriving at the portion of the metal foil; and
pumping the gas into the housing so that the laser beam and the gas exit from the housing at the same output.

9. The method of claim 8, wherein the output of the housing guides the laser beam and the gas directly onto the portion of the metal foil that needs to be melted.

10. The method of claim 8, further comprising:
adjusting a position of a lens, inside the housing, to focus the laser beam onto the portion of the metal foil.

11. A microfluidic platform for growing cells, the microfluidic platform comprising:
a silicon wafer having microfluidic passages in which the cells grow;
a glass layer formed directly on a first face of the silicon wafer;
a first indium tin oxide, ITO, layer formed directly on the glass layer, opposite to the silicon wafer; and
first and second metal pads form directly on the first ITO layer,
wherein the first and second metal pads are connected to a power source so that the first ITO layer acts as a heater for heating the microfluidic passages.

12. The microfluidic platform of claim 11, further comprising:
a layer of polydimethylsiloxane, PDMS, directly formed on a second face of the silicon wafer, which is opposite to the first face;
a second ITO layer formed on the PDMS layer; and
a glass layer formed over the second ITO layer.

13. The microfluidic platform of claim 12, further comprising:
metal pads directly formed on the second ITO layer.

14. The microfluidic platform of claim 12, further comprising:
at least one sensor formed in the second ITO layer.

15. The microfluidic platform of claim 12, wherein the entire structure is transparent to electromagnetic waves so that imagining processes can be used to view the cells.

16. The microfluidic platform of claim 12, further comprising:
a microporous membrane placed between an upper portion of the silicon wafer and a lower portion of the silicon waver.

17. The microfluidic platform of claim 16, wherein the microporous membrane separates the cells from a flow of fluid.

18. A method of making a microfluidic platform that is entirely transparent to electromagnetic waves, the method comprising:
forming microfluidic passages in a silicon wafer;
forming a first indium tin oxide, ITO, layer directly on a first glass layer;
attaching the first glass layer directly to a first face of the silicon wafer; and
forming first and second metal pads directly onto the first ITO layer.

19. The method of claim 18, further comprising:
forming a second ITO layer onto a second glass layer;
attaching a layer of polydimethylsiloxane, PDMS, directly to a second face of the silicon wafer, which is opposite to the first face; and
attaching the second ITO layer directly to the PDMS layer.

20. The method of claim 19, further comprising:
forming metal pads directly onto the second ITO layer by melting a metal foil on the second ITO layer with a laser beam while pumping an inert gas where the inert gas interacts with the metal foil to prevent oxidation.
